(19) United States
(12) Plant Patent Application Publication
(10) Pub. No.: US 2023/0389456 P1
(43) Pub. Date: Nov. 30, 2023

(54) Strawberry Plant Named 'LBR 96 1'

(50) ;

(71) Applicant: INSTITUTO NACIONAL DE INVESTIGACION AGROPECUARIA, Montevideo (UY)

(72) Inventors: Gustavo Glménez, Canelones (UY); Alberto Lenzi, Canelones (UY)

(21) Appl. No.: 17/803,542

(22) Filed: Aug. 17, 2022

Related U.S. Application Data
(60) Provisional application No. 63/259,998, filed on Aug. 27, 2021.

(51) Int. Cl. A01H 6/74

(52) U.S. Cl. PLT/208

(57) ABSTRACT

A new day neutral strawberry plant named ‘LBR 96 1’ is disclosed, with remarkable fruit and flavor characteristics and high levels of resistance to pest and diseases.

### CROSS-REFERENCE TO RELATED APPLICATIONS

This application claims priority of co-pending provisional application Ser. No. 63/259,998 as filed on Aug. 27, 2021.

### STATEMENT REGARDING FEDERALLY SPONSORED RESEARCH OR DEVELOPMENT

None

Genus and species: Fragaria X ananassa Duch.

Variety denomination: ‘LBR 96 1’.

### BACKGROUND OF THE INVENTION

‘LBR 96 1’ was obtained from the INSTITUTO NACIONAL DE INVESTIGACION AGROPECUARIA (National Institute for Agricultural Research) a breeding program located in Canelones, Uruguay. It originated from the cross between two advanced clones that was carried out in 2012 at the INIA Las Brujas facility in Canelones, Uruguay. It was selected in from a field of individuals in 2014 and later evaluated in preliminary and advanced agronomic trials against reference varieties, always under conditions representative of the production technology of the Salto area, Uruguay.

‘LBR 96 1’ was simultaneously characterized by its sanitary behavior against the natural infection of root/crown, foliar, and fruit pathogens (2014-2019). During 2018 and 2019, it was evaluated at the productive level in the producer's farms. During the last three years physical, chemical, and quality of fruit has been characterized. In 2020, the cultivar was registered, protected, and licensed.

Compared to its maternal parent ‘LBK 36.1A’ (INIA Mayte™), ‘LBR 96 1’ is more tolerant to soil, foliar and fruit diseases with superior fruit quality and agronomic behavior. According to the information available, ‘LBR 96 1’ would be recommended for the south production region of Uruguay.

The new cultivar ‘LBR 96 1’ has a superior performance than ‘LBK36.1A’ in early productivity (harvests July-September) and total production (harvest autumn, spring, and summer); in fruit quality, especially flavor; and health, due to its lower incidence of botrytis and powdery mildew in leaf and fruit.

### BRIEF DESCRIPTION OF THE PHOTOGRAPH

FIG. 1 illustrates the fruit of ‘LBR 96 1’ harvested July 2021 from a 5-month-old plant;

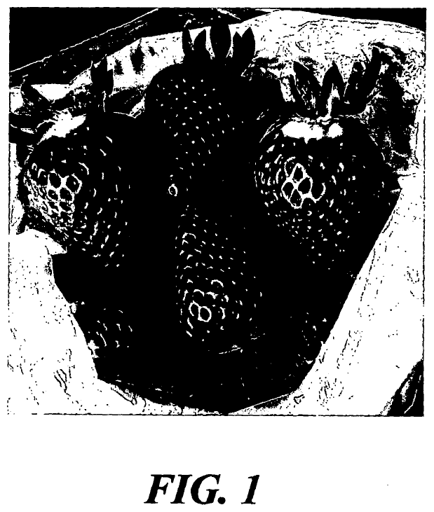

FIG. 2 illustrates plants of ‘LBR 96 1’ at 5 months of age cultivated with black plastic mulch and drip irrigation;

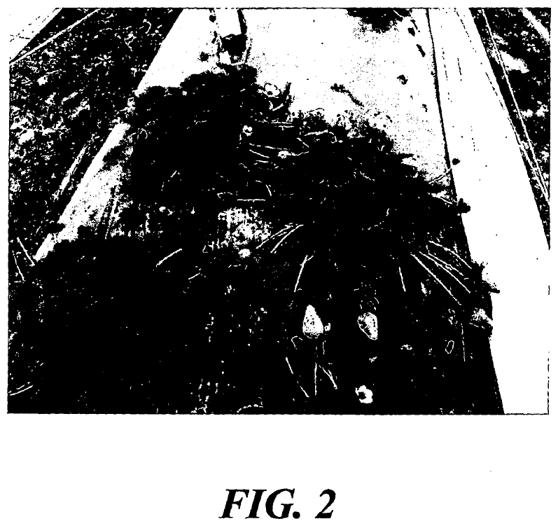

FIG. 3 illustrates plants of ‘LBR 96 1’ at 5 months of age with ripe fruit just before harvest; and

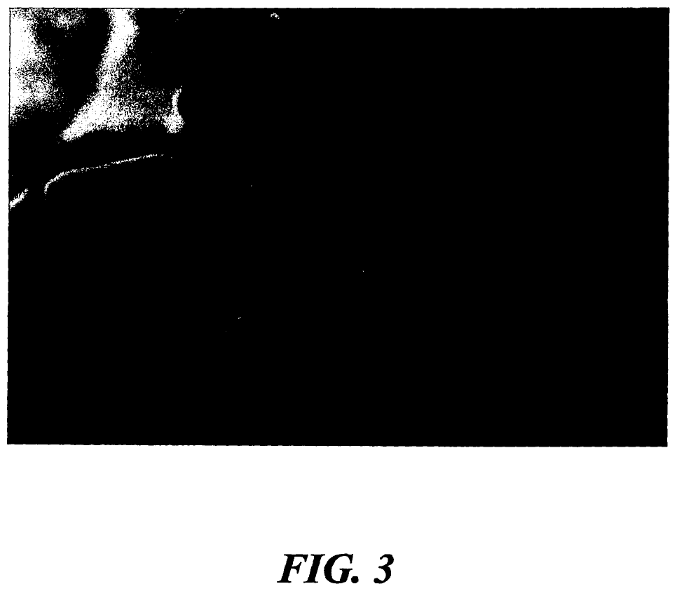

FIG. 4 illustrates production of stolons undergreenhouse with drip irrigation.

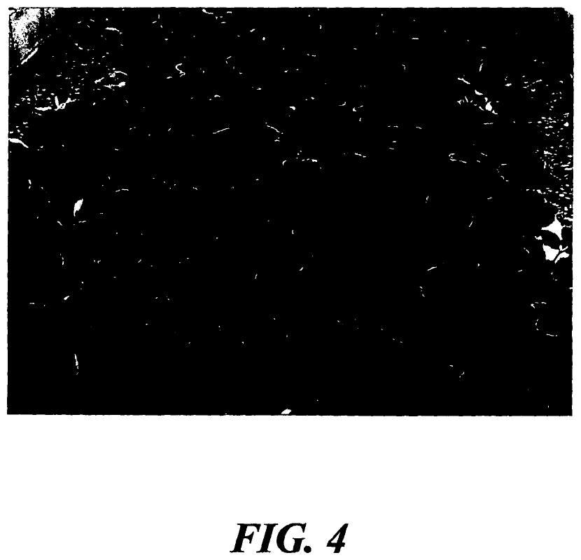

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2021 growing seasons in INIA Las Brujas, Canelones, Uruguay. It should be understood that the characteristics described will vary somewhat depending upon cultural practices and climatic conditions and can vary with location and season. Measurements are the average for the plants of the new cultivar and may vary according to the stage of development.
General description:
   Ploidy.—Octoploid.
   Blooming period.—April thru March in the Southern Hemisphere.
   Plant type.—Day Neutral.
   Plant habit.—Erect.
   Root description.—Cream white to white in color.
   Propagation.—By runners tips, cutting and direct rooting in trays.
   Growth rate.—Medium vigor.
   Stolon description.—Number of stolons: medium;
   Anthocyanin.—Weak or null.
Foliage description:
   Leaf division.—Typically three leaflets per leaf.
   Leaf blistering.—Medium.
   Leaf glossiness.—Medium.
   Leaf variegation.—Absent.
   Leaf arrangement.—Spiral arrangement.
   Leaflet shape in cross section.—Concave.
   Leaflet length in relation to width.—Longer.
   Leaflet margins.—Crenate.
   Leaflet base.—Obtuse.
   Leaflet apex.—Rounded.
   Leaflet color.—Green.
   Petiole.—Length medium:
Flower description:
   Position of inflorescence in relation to foliage.—Higher than foliage.
   Flower initiation and expression conditions.—Very low to no cold requirements to flower; early cultivar of day neutral.
   Time of flowering (50% of plants at first flower).—Early, in Uruguay they bloom year around from April to March of the following year.
   Size of calyx in relation to corolla.—Larger position of calyx attachment.
   Petals.—Length in relation to width: moderately longer; color of upper side: white.
Fruit description:
   Shape.—Conical.
   Season of harvest.—May thru March.
   Time of ripening (50% of plants with first ripe fruit).—Very early; in Uruguay harvest begins in Mid-May.
   Time of bearing.—25-30 days depending on weather conditions and season of the year.
   Size.—Large to very large.
   Evenness of surface.—Even or very slightly uneven.
   Glossiness.—High gloss, great fruit appearance.
   External color (skin).—Red.
   Internal color.—Light red.
   Evenness of color of skin.—Even distribution.
   Acidity.—0.5-0.65 Titratable acidity (% citric acid).
   Sweetness.—7.5-8.6 brix.
   Firmness.—very firm 0.8-0.9 N.
   Fruit weight.—16 g-25 g.
   Shelf life.—Very good postharvest life.
   Achene.—Level with surface; Width of band without achenes: very narrow.
Disease and pest resistance:
   Global response to the disease complex root and stem.—High field tolerance.
   Resistance to powdery mildew at leaf level.—Medium-High.
   Resistance to powdery mildew at fruit level.—Medium-High.
   Resistance to botrytis.—Low incidence.
   Resistance to spider mites.—Low preference.
   Resistance to anthracnose in fruit.—High.

1. A new and distinct variety of strawberry plant named ‘LBR 96 1’, substantially as illustrated and described herein.
